SEQUENCE LISTING (11 sequences)

SEQ ID NO: 1 - DNA, 21 bp, synthetic construct
  source 1..21: mol_type = other DNA
taaagttcgtttagagaacag

SEQ ID NO: 2 - DNA, 24 bp, synthetic construct
  source 1..24: mol_type = other DNA
cctgggaaggtataaacctttaat

SEQ ID NO: 3 - DNA, 20 bp, synthetic construct
  source 1..20: mol_type = other DNA
ttagagaacagatctacaag

SEQ ID NO: 4 - DNA, 20 bp, synthetic construct
  source 1..20: mol_type = other DNA
cagatctacaagagatcgaa

SEQ ID NO: 5 - DNA, 24 bp, synthetic construct
  source 1..24: mol_type = other DNA
gttcgtttagagaacagatctaca

SEQ ID NO: 6 - DNA, 24 bp, synthetic construct
  source 1..24: mol_type = other DNA
ttagagaacagatctacaagagat

SEQ ID NO: 7 - DNA, 24 bp, synthetic construct
  source 1..24: mol_type = other DNA
cagatctacaagagatcgaaagtt

SEQ ID NO: 8 - DNA, 22 bp, synthetic construct
  source 1..22: mol_type = other DNA
gtgaratggtcatgtgtggcgg

SEQ ID NO: 9 - DNA, 26 bp, synthetic construct
  source 1..26: mol_type = other DNA
caratgttaaasacactattagcata

SEQ ID NO: 10 - DNA, 25 bp, synthetic construct
  source 1..25: mol_type = other DNA
caggtggaacctcatcaggagatgc

SEQ ID NO: 11 - RNA, 80 nt, Severe acute respiratory syndrome coronavirus 2
  source 1..80: mol_type = genomic RNA
attaaaggtttataccttcccaggtaacaaaccaaccaactttcgatctcttgtagatctgttctctaaacgaactttaa

(19) United States
(12) Patent Application Publication
(10) Pub. No.: US 2024/0417733 A1
(43) Pub. Date: Dec. 19, 2024

(54) TIS-L TARGETING ANTIVIRAL ANTISENSE OLIGONUCLEOTIDE WITH ABILITY TO INHIBIT VIRAL REPLICATION OF SARS-COV-2

(71) Applicants: SEOUL NATIONAL UNIVERSITY R&DB FOUNDATION, Seoul (KR); Korea University Research and Business Foundation, Seoul (KR); KOREA ADVANCED INSTITUTE OF SCIENCE AND TECHNOLOGY, Daejeon (KR); Korea National Institute of Health, Cheongju-si (KR)

(72) Inventors: Daehyun BAEK, Seoul (KR); Hee Ryung CHANG, Seoul (KR); Jeong-Sun YANG, Cheongju-si (KR); Hansaem LEE, Cheongju-si (KR); Man-Seong PARK, Seoul (KR); Heedo PARK, Seoul (KR); Yoon Ki KIM, Daejeon (KR); Jeeyoon CHANG, Daejeon (KR); Joori PARK, Seoul (KR); Joo-Yeon LEE, Cheongju-si (KR); Kyung-Chang KIM, Cheongju-si (KR)

(73) Assignees: SEOUL NATIONAL UNIVERSITY R&DB FOUNDATION, Seoul (KR); Korea University Research and Business Foundation, Seoul (KR); KOREA ADVANCED INSTITUTE OF SCIENCE AND TECHNOLOGY, Daejeon (KR); Korea National Institute of Health, Cheongju-si (KR)

(21) Appl. No.: 18/744,136

(22) Filed: Jun. 14, 2024

(30) Foreign Application Priority Data
Jun. 16, 2023 (KR) 10-2023-0077680

(51) Int. Cl. C12N 15/113; A61P 31/14

(52) U.S. Cl. CPC C12N 15/1131; A61P 31/14; C12N 2310/11; C12N 2310/321; C12N 2310/3231

(57) ABSTRACT

The present disclosure relates to a TIS-L targeting antisense oligonucleotide that inhibits the viral replication of SARS-COV-2 and antiviral use thereof. Since the TIS-L targeting antisense oligonucleotide according to the present disclosure has a simple structure, it is easy to mass produce, and as it exhibits antiviral efficacy of 90% or more even at low doses against the original strain and various modified strains of the virus, it can be advantageously utilized in the development of COVID-19 treatment.

### CROSS-REFERENCE TO RELATED APPLICATION

This application claims priority to and the benefit of Korean Patent Application No. 10-2023-0077680, filed on Jun. 16, 2023, the disclosure of which is incorporated herein by reference in its entirety.

### INCORPORATION BY REFERENCE OF SEQUENCE LISTING

The content of the electronically submitted sequence listing, file name: Q299270_sequence listing as filed. XML; size: 10,702 bytes; and date of creation: Jun. 14, 2024, filed herewith, is incorporated herein by reference in its entirety.

### BACKGROUND

### 1. Field of the Invention

The present disclosure relates to a TIS-L targeting antisense oligonucleotide that inhibits the viral replication of SARS-COV-2 and antiviral use thereof.

### 2. Discussion of Related Art

Severe acute respiratory syndrome coronavirus 2 (SARS-COV-2) is a betacoronavirus, and it is classified in the same family as 6 other highly contagious human coronaviruses (229E, OC43, HKU1, SARS-COV and MERS-COV). Human coronaviruses HCoV229E, OC43, NL63 and HKU1, which are known to be pathogenic in humans, cause relatively mild respiratory disease, whereas HCoVs SARS-COV, MERS-COV and SARS-COV-2 caused serious outbreaks with fatal symptoms in 2002, 2012 and 2019. In particular, SARS-COV-2 has caused an epidemic of unprecedented scale, leading the United Nations to declare it a pandemic and having a huge impact on society and the economy globally.

SARS-COV-2 has a structure in which an envelope surrounds a protein capsid containing a 30 kb single-stranded positive sense RNA genome that is used as a template for transcription, and the spike proteins in the form of a membrane glycoprotein protrude like protrusions on the surface. Among these, the spike protein binds to the surface of a receptor present on the host, which protrudes like a nail, and plays the role of penetrating into the host cell, so as to perform an important role of infecting the host with the virus. The spike protein is functionally divided into an S1 subunit that binds to the receptor of the host cell and an S2 subunit that fuses to the membrane, and in the S1 subunit, a receptor binding domain (RBD) that binds to the host cell's ACE2 (Angiotensin converting enzyme 2) receptor exists. The RNA genome consists of ORFs 1a and 1b, two-thirds of which encode pp1a (polyprotein 1a) and pp1ab at the 5′ end, and these undergo post-translational processes into 16 non-structural proteins. The remaining genome encodes for structural and accessory proteins, and the composition thereof varies depending on the coronavirus. According to a recent study, the complete genome structure of SARS-COV-2 includes ORF1a, 1b, structural proteins, spike(S), envelope (E), membrane (M), nucleocapsid (N), 3a, 6, 7a, 7b, 8 and 10. ORF1a and 1b are translated directly from gRNA, whereas the remaining ORFs are translated from sgRNA. All sgRNAs have overlapping positive-strand RNA ends with the 5′ end capped and the 3′ end polyadenylated.

The 5′ end of the gene of the SARS-COV-2 virus has a leader sequence including TRS-L (transcription regulatory sequence-leader) at the 3′ end. TRS is located upstream of each ORF (TRS-body; TRS-B), and the 6-nt core sequence is identical to all TRSs. The 5′ end of each ORF is fused to the leader sequence through discontinuous transcription by the RNA-dependent RNA polymerase complex encoded by ORF1a and 1b. Viral genome replication and transcription are performed by viral proteins, whereas the translation process is performed by host proteins. Therefore, observing the dynamics of the virus and the human transcriptome is very important in understanding the molecular mechanism of Coronavirus Disease 2019 (hereinafter, COVID-19) pathogenicity in humans, which can be used to develop new drugs.

The inventors of the present disclosure confirmed the genome structure and gene expression pattern of SARS-COV-2 and identified a translation regulator (Translation initiation site located in the leader sequence; TIS-L) (Korean Patent Application Laid-Open No. 10-2023-0030690, NATURE COMMUNICATIONS|(2021) 12:5120), and predicted that when the antisense oligonucleotide (ASO), which inhibits the function of TIS-L, is administered, it is possible to effectively treat COVID-19 by disrupting the overall gene expression of SARS-COV-2 and greatly reducing the viral replication and infectivity of the virus. In the present disclosure, an antisense oligonucleotide that showed a particularly remarkable effect among various antisense oligonucleotide candidates that inhibit the function of TIS-L was developed, and the present disclosure was completed by verifying the effects thereof.

### RELATED ART DOCUMENTS

### Patent Documents

(Patent Document 1) Korean Patent Application Laid-Open No. 10-2023-0030690

### Non-Patent Documents

(Non-Patent Document 1) NATURE COMMUNICATIONS|(2021) 12:5120

### SUMMARY OF THE INVENTION

An object of the present disclosure is to provide a TIS-L targeting antisense oligonucleotide that inhibits the viral replication of various mutant strains of SARS-CoV-2 and antiviral use thereof.

In order to achieve the above object, the present disclosure provides an oligonucleotide represented by the nucleotide sequence of SEQ ID NO: 5 or SEQ ID NO: 7.

In the present disclosure, the oligonucleotide may be for inhibiting the viral replication of SARS-COV-2.

In the present disclosure, the oligonucleotide may be an antisense oligonucleotide that targets TIS-L.

In the present disclosure, the oligonucleotide represented by the nucleotide sequence of SEQ ID NO: 7 may exert an inhibitory effect on the viral replication of the original strain and mutant strains of SARS-COV-2.

In the present disclosure, the oligonucleotide may consist of DNA nucleotides, RNA nucleotides, PNA nucleotides, LNA nucleotides, PMO nucleotides, PS nucleotides, 2′-O-Me nucleotides, 2′-O-MOE nucleotides or any combination thereof. In the present disclosure, the oligonucleotide may consist of a combination of DNA nucleotides and LNA nucleotides.

In the present disclosure, in the oligonucleotide, the 1^{st} to 3^{rd} nucleotides and the 22^{nd} to 24^{th} nucleotides of SEQ ID NO: 5 or SEQ ID NO: 7 may be LNA, and the 4^{th} to 21^{st} nucleotides may be DNA.

In addition, the present disclosure provides a pharmaceutical composition for preventing or treating COVID-19, including the oligonucleotide or a pharmaceutically acceptable salt of the oligonucleotide as an active ingredient.

In the present disclosure, the pharmaceutical composition may further include a carrier.

In the present disclosure, the carrier may be selected from the group consisting of viral vectors, non-viral vectors, liposomes, cationic polymers, micelles, emulsions, solid lipid nanoparticles and exosomes.

In addition, the present disclosure provides a method for preventing or treating COVID-19, comprising administering the oligonucleotide or a pharmaceutically acceptable salt of the oligonucleotide to a subject in need thereof.

In the present disclosure, wherein the oligonucleotide or a pharmaceutically acceptable salt of the oligonucleotide is administered via a carrier.

In the present disclosure, wherein the carrier is selected from the group consisting of viral vectors, non-viral vectors, liposomes, cationic polymers, micelles, emulsions, solid lipid nanoparticles and exosomes.

Since the TIS-L targeting antisense oligonucleotide according to the present disclosure has a simple structure, it is easy to mass produce, and as it exhibits antiviral efficacy of 90% or more even at low doses against the original strain and various modified strains of the virus, it can be advantageously utilized in the development of COVID-19 treatment.

### BRIEF DESCRIPTION OF THE DRAWINGS

The above and other objects, features and advantages of the present disclosure will become more apparent to those of ordinary skill in the art by describing in detail exemplary embodiments thereof with reference to the accompanying drawings, in which:

FIG. 1A is a mimetic diagram showing the target sequences of an antisense oligonucleotide (Neuman sequence) targeting TIS-L and an antisense oligonucleotide (Vora sequence) showing SARS-COV-2 inhibitory effect that are searched through the literature;

FIG. 1B is a mimetic diagram showing qRT-PCR and plaque assay experimental methods to confirm the inhibitory effect of the viral replication of SARS-CoV-2 according to antisense oligonucleotide treatment;

FIG. 1C is the results of confirming the inhibitory effect of the Neuman sequence and the Vora sequence on the viral replication of SARS-COV-2 by plaque assay;

FIG. 2A is a mimetic diagram showing the target sequences of two types of 20 mer antisense oligonucleotides (MIDDLE-(20 mer) sequence and END-(20 mer) sequence) targeting TIS-L;

FIG. 2B is the results of confirming the inhibitory effect of the MIDDLE-(20 mer) sequence and END-(20 mer) sequence on the viral replication of SARS-COV-2 by plaque assay;

FIG. 3A is a mimetic diagram showing the target sequences of three types of 24 mer antisense oligonucleotides (BOTH-(24 mer) sequence, MIDDLE-(24 mer) sequence and END-(24 mer)) targeting TIS-L;

FIG. 3B is the results of confirming the inhibitory effect of the BOTH-(24 mer) sequence, MIDDLE-(24 mer) sequence and END-(24 mer) sequence on the viral replication of the original SARS-COV-2 strain by plaque assay;

FIG. 3C is the results of qRT-PCR confirming the inhibitory effect of the BOTH-(24 mer) sequence, MIDDLE-(24 mer) sequence and END-(24 mer) sequence on the viral replication of the original strain and mutant strains of SARS-COV-2;

FIG. 4 is the results of confirming the IC₅₀ of END-(24 mer) antisense oligonucleotide against the original strain and various mutant strains of SARS-COV-2 by qRT-PCR;

FIG. 5A is a mimetic diagram showing the WST-1 Assay experiment method; and

FIG. 5B is the results of WST-1 Assay confirming the cytotoxicity of END-(24 mer) antisense oligonucleotide by using Vero cell line.

FIG. 6 is the results of quantifying viral titers using a plaque assay. Organs (lung, nasal turbinates) were excised 1-4 days post-infection, homogenized with culture medium, and the supernatant was collected.

### DETAILED DESCRIPTION OF EXEMPLARY EMBODIMENTS

Unless otherwise defined, all technical and scientific terms used in the present specification have the same meanings as commonly understood by a person skilled in the art to which the present disclosure pertains. In general, the nomenclature used herein is well known and commonly used in the art.

In the present disclosure, the inventors of the present disclosure sought to develop an antisense oligonucleotide (ASO) that exerts an inhibitory effect on the viral replication of SARS-COV-2, which is the virus that caused COVID-19 and has caused a pandemic since 2019, and as a result of designing the ASO which consists of DNA and/or LNA targeting a translation regulator (Translation initiation site located in the leader sequence; TIS-L), which is expected to have a significant impact on the overall expression of the SARS-COV-2 gene identified in previous studies, it was confirmed that the antisense oligonucleotide having a specific sequence had a inhibition efficacy of 90% or more on the viral replication of the original SARS-COV-2 strain and various mutant viruses.

Accordingly, in one aspect, the present disclosure relates to an oligonucleotide represented by the nucleotide sequence of SEQ ID NO: 5 or SEQ ID NO: 7.

In the present disclosure, the oligonucleotide may be for inhibiting the viral replication of SARS-COV-2.

In the present disclosure, the oligonucleotide may be an antisense oligonucleotide that targets TIS-L.

In the present disclosure, particularly, the oligonucleotide represented by the nucleotide sequence of SEQ ID NO: 7 may exhibit an effect of inhibiting the viral replication of the original strain and mutant strains of SARS-COV-2.

In the present disclosure, the oligonucleotide may consist of DNA nucleotides, RNA nucleotides, PNA nucleotides, LNA nucleotides, PMO nucleotides, PS nucleotides, 2′-O-Me nucleotides, 2′-O-MOE nucleotides or any combination thereof.

In one aspect, the oligonucleotide may consist a combination of DNA nucleotides and LNA nucleotides.

For example, the oligonucleotide may be characterized in that the 1^{st} to 3^{rd} and 22^{nd} to 24^{th} nucleotides of SEQ ID NO: 5 or SEQ ID NO: 7 are LNA, and the 4^{th} to 21^{st} nucleotides are DNA.

The oligonucleotide according to the present disclosure has the advantage of being easy to mass produce because it is not long and has a simple structure that consists of DNA and LNA.

The oligonucleotide according to the present disclosure may target TIS-L.

Since TIS-L is located in the SARS-COV-2 leader sequence and around the stem loop (SL), which is an RNA structure, common sense would develop an antisense oligonucleotide, but it is not considered as a suitable target sequence in this technical field.

Accordingly, in general, when selecting the target site of an antisense oligonucleotide, secondary structure is taken into consideration, and a position without a structure is selected as the target site of the antisense oligonucleotide. For this purpose, RNA secondary structure calculation software such as m-fold is used (Aartsma-Rus, Annemieke, et al. “Guidelines for antisense oligonucleotide design and insight into splice-modulating mechanisms.” Molecular Therapy 17.3 (2009): 548-553).

Actually, in the present disclosure, as a result of comparing the inhibitory effect of antisense oligonucleotides on the viral replication of SARS-COV-2 as disclosed in prior literature (Vora, Setu M., et al. “Targeting stem-loop 1 of the SARS-CoV-2 5′ to suppress viral translation and Nsp1 evasion.” Proceedings of the National Academy of Sciences 119.9 (2022): e2117198119) targeting TIS-L with the inhibitory effect of antisense oligonucleotides on the viral replication of SARS-COV-2 as disclosed in other prior literature (Neuman, Benjamin W., et al. “Inhibition, escape, and attenuated growth of severe acute respiratory syndrome coronavirus treated with antisense morpholino oligomers.” Journal of Virology 79.15 (2005): 9665-9676) not targeting TIS-L, it was confirmed that the antisense oligonucleotide targeting TIS-L showed a relatively low effect in inhibiting the viral replication of SARS-COV-2.

Additionally, in the results of confirming the inhibition effects on the viral replication of SARS-COV-2 by designing an antisense oligonucleotide targeting 20-mer TIS-L, which is the length commonly used in antisense oligonucleotides, it showed that antisense oligonucleotides targeting 20 mer TIS-L did not effectively inhibit the viral replication of SARS-COV-2.

On the other hand, the antisense oligonucleotide (END-(24 mer)) of the present disclosure, which had a length of 24 mer and was designed to bind complementarily to TIS-L at the 3′ end, was not only able to very effectively inhibit the viral replication of SARS-COV-2, but also the antisense oligonucleotide was significantly effective in inhibiting viral replication not only in the original strain of SARS-COV-2 but also in various mutant strains.

In the present disclosure, the term “antisense oligonucleotide” or “antisense compound” refers to RNA, DNA, LNA, PNA or a mixture of molecules thereof that bind to other RNA or DNA (target RNA, DNA). For example, if it is an RNA oligonucleotide, it binds to another RNA target by RNA-RNA interaction and alters the activity of the target RNA. Antisense oligonucleotides can up-regulate or down-regulate the expression and/or function of specific polynucleotides. This definition is intended to include any foreign RNA or DNA molecule that is useful from a therapeutic, diagnostic or other perspective. These molecules include, for example, antisense RNA or DNA molecules, interfering RNA (RNAi), micro RNA, bait RNA molecules, siRNA, enzyme RNA, therapeutic editing RNA and antisense oligomeric compounds, antisense oligonucleotides, external guide sequences (EGS) oligonucleotides, primers, probes and other oligomeric compounds that hybridize to at least a portion of the target nucleic acid. Accordingly, these compounds may be introduced in the form of single-stranded, double-stranded, partially single-stranded or circular oligomeric compounds.

In the context of the present disclosure, the term “oligonucleotide” refers to an oligomer or polymer of ribonucleic acid (RNA) or deoxyribonucleic acid (DNA), or a mimic thereof. The term “oligonucleotide” also includes natural and/or modified monomers or chains of linear or circular oligomers, such as deoxyribonucleosides, ribonucleosides, substituted and alpha-anomeric forms thereof, peptide nucleic acids (PNA), locked nucleic acids (LNA), phosphorothioate, methylphosphonate. Oligonucleotides can specifically bind to target polynucleotides by the regular patterns of monomer-monomer interactions, such as Watson-Crick type base pairing, Hoogsteen or reverse Hoogsteen type base pairing and the like.

Oligonucleotides may be “chimeric,” that is, they may be composed of different regions. In the context of the present disclosure, a “chimeric” compound may be an oligonucleotide possessing two or more chemical regions, for examples, DNA region(s), RNA region(s), PNA region(s), LNA region(s) and the like.

Oligonucleotides may be composed of regions that can be linked when monomers are linked sequentially, as in native DNA, or linked through spacers. These spacers constitute a covalent “bridge” between these regions and, where desired, are intended to have a length not exceeding approximately 100 carbon atoms. These spacers, for example, may possess a positive or negative charge, have specific nucleic acid binding properties (inserts, groove binders, toxins, fluorophores, etc.), be lipophilic, and for example, possess different functionalities that induce specific secondary structures, such as alanine-containing peptides that induce alpha-helix formation.

As used herein, the term “target nucleic acid” encompasses DNA, RNA transcribed from such DNA (including pre-mRNA and mRNA) and cDNA derived from such RNA, which encodes non-coding sequences, sense or antisense polynucleotides. Specific hybridization of the oligomeric compound with the target nucleic acid interferes with the normal function of the nucleic acid. This modulation of the function of a target nucleic acid by a compound that specifically hybridizes to the target nucleic acid is generally referred to as “antisense.” The functions of DNA that are interfered with include, for example, replication and transcription.

RNA interference “RNAi” is mediated by double-stranded RNA (dsRNA) molecules that have sequence-specific homology to a “target” nucleic acid sequence. In certain embodiments of the present disclosure, the mediator is a 5 to 25 nucleotide “small interfering” RNA duplex (siRNA). These siRNAs are derived from the processing of dsRNA by the RNase enzyme known as Dicer. The siRNA duplex product is recruited into a multi-protein siRNA complex named RISC (RNA Induced Silencing Complex). Without being restricted by a particular theory, it is believed that RISC is then guided to a target nucleic acid (appropriately an mRNA), where the siRNA duplex interacts in a sequence-specific manner to catalytically mediate cleavage. Small interfering RNAs that can be used in accordance with the present disclosure may be synthesized and used according to procedures well known in the art and familiar to those skilled in the art. The small interfering RNA used in the methods of the present disclosure suitably includes approximately 1 to approximately 50 nucleotides (nt). In an illustrative example of a non-limiting embodiment, the siRNA may include approximately 5 to approximately 40 nt, approximately 5 to approximately 30 nt, approximately 10 to approximately 30 nt, approximately 15 to approximately 25 nt, or approximately 20 to 25 nucleotides.

The term “nucleotide” includes naturally occurring nucleotides and non-naturally occurring nucleotides. It will be clear to those skilled in the art that various nucleotides that were previously considered to be “non-naturally occurring” are subsequently being discovered in nature. Accordingly, “nucleotide” includes not only the known purine and pyrimidine heterocycle-bearing molecules, but also the heterocyclic analogs and tautomers thereof. Illustrative examples of different types of nucleotides are molecules that possess adenine, guanine, thymine, cytosine, uracil, purine, xanthine, diaminopurine, 8-oxo-N6-methyladenine, 7-deazaxanthin, 7-deazaguanine, N4, N4-ethanocytosine, N6, N6-ethano-2,6-diaminopurine, 5-methylcytosine, 5-(C3-C6)-alkynylcytosine, 5-fluorouracil, 5-bromouracil, pseudocytosine, 2-hydroxy-5-methyl-4-triazolopyridine, isocytosine, isoguanine, inosine and “non-naturally occurring” nucleotides described Benner et al and U.S. Pat. No. 5,432,272.

The term “nucleotide” may include all of these examples, as well as the analogs and tautomers thereof. The nucleotides of particular interest are those containing adenine, guanine, thymine, cytosine and uracil, which are considered as naturally occurring nucleotides with respect to therapeutic and diagnostic applications in humans. Nucleotides include, for example, natural 2′-deoxy and 2′-hydroxyl sugars, and the analogs thereof described in in Kornberg and Baker, DNA Replication, 2^{nd} Ed. (Freeman, San Francisco, 1992).

With respect to nucleotides, the “analogs” include synthetic nucleotides bearing modified base moieties and/or modified sugar moieties (e.g., described generally in Scheit, Nucleotide Analogs, John Wiley, New York, 1980; Freier & Altmann, (1997) Nucl. Acid. Res., 25 (22), 4429-4443, Toulme J. J., (2001) Nature Biotechnology 19:17-18; Manoharan M., (1999) Biochemica et Biophysica Acta 1489:117-139; Freier S. M., (1997) Nucleic Acid Research, 25:4429-4443, Uhlman, E., (2000) Drug Discovery & Development, 3:203-213, Herdewin P., (2000) Antisense & Nucleic Acid Drug Dev., 10:297-310); 2′-O, 3′-C-linked [3.2.0] bicycloarabinonucleosides). These analogs include synthetic nucleotides that are designed to enhance binding properties, such as double or triple helix stability, specificity and the like.

As used herein, the term “complementarity” refers to the ability of correct pairing between two nucleotides in one or two oligomeric strands. For example, if a nucleobase at a certain position of an antisense compound can form a hydrogen bond with a nucleobase at a certain position of a target nucleic acid, and the target nucleic acid is a DNA, RNA or oligonucleotide molecule, the position of the hydrogen bond between the oligonucleotide and the target nucleic acid is considered to be a complementary position. An oligomeric compound and an additional DNA, RNA or oligonucleotide molecule are complementary to each other, when a sufficient number of complementary positions within each molecule are occupied by nucleotides that can hydrogen bond to each other. Accordingly, “specifically hybridizable” and “complementarity” are terms used to indicate a sufficient degree of precise pairing or complementarity over a sufficient number of nucleotides to cause stable and specific binding between an oligomeric compound and a target nucleic acid.

In the relevant field, the sequence of an oligomeric compound does not need to be 100% complementary to the sequence of the target nucleic acid in order to hybridize specifically. In addition, oligonucleotides may hybridize across one or more segments (e.g., loop structures, mismatch or hairpin structures) such that intervening or adjacent segments are not involved in the hybridization event. The oligomeric compounds of the present disclosure include at least approximately 70%, or at least approximately 75%, or at least approximately 80%, or at least approximately 85%, or at least approximately 90%, or at least approximately 95%, or at least approximately 99% sequence complementarity of the target region within the target nucleic acid sequence to which they are targeted. For example, 18 of the 20 nucleotides of an antisense compound may be complementary to the target region, and therefore, an antisense compound that hybridizes specifically will exhibit 90% complementarity. In these examples, the remaining non-complementary nucleotides may be clustered together with the complementary nucleotides, or the complementary nucleotides may be interspersed, and they do not need be adjacent to each other or to complementary nucleotides. Accordingly, an antisense compound of 18 nucleotides in length with 4 non-complementary nucleotides which are flanked by two regions of complete complementarity with the target nucleic acid would have 77.8% overall complementarity with the target nucleic acid, and would therefore fall within the scope of the present disclosure. The complementarity ratio of the antisense compound with the region of the target nucleic acid may be routinely determined by using the BLAST (basic local alignment search tools) and the PowerBLAST program that are known in the art. Percent homology, sequence identity or complementarity may be determined, for example, by the Gap program (Wisconsin Sequence Analysis Package, Version 8 for Unix, Genetics Computer Group, University Research Park, Madison Wis.) by using default settings, and it uses the algorithm of Smith and Waterman, Adv. Appl. Math., (1981) 2, 482-489.

The oligonucleotides of the present disclosure may be of any chemical or natural modification. Chemical and natural modifications are well known in the art. These modifications include, for example, modifications that are designed to improve the pharmacokinetic properties of oligonucleotides to increase binding to the target strands (i.e., increase their melting temperature), to assist in the identification of oligonucleotides or oligonucleotide-target complexes, to increase cell penetration, to stabilize against nucleases and other enzymes that degrade or interfere with the structure or activity of the oligonucleotide, and to provide a mode of termination (termination event) upon sequence-specific binding to the target. Modifications include, for example: (a) terminal modifications, such as 5′ end modifications (phosphorylation-dephosphorylation, splicing, inversion ligation, etc.), 3′ end modifications (splicing, DNA nucleotides, inversion ligation, etc.), (b) base modifications, for example, a modified base, a stabilizing base, a destabilizing base or a base that forms base pairs with an expanded repertoire of partners, or replacement with a conjugated base, and (c) a sugar modification (e.g., at the 2′ position or at the 4′ position) or sugar replacements, as well as (d) internucleoside linkage modifications, such as modifications or replacements of phosphodiester linkages, but the present disclosure is not limited thereto. To the extent that such modifications interfere with translation (i.e., resulting in a 50%, 60%, 70%, 80% or 90% or greater reduction in translation compared to no modification, e.g., an in vitro translation assay), modifications may not be optimal for the methods and compositions described herein.

Non-limiting examples of modified internucleoside linkages include phosphorothioates with normal 3′-5′ linkages, chiral phosphorothioates, phosphorodithioates, phosphotriesters, aminoalkylphosphotriesters, methyl and other alkyl phosphonates, such as 3′-alkylene phosphonates and chiral phosphonates, phosphinates, phosphoramidates, such as 3′-amino phosphoramidate and aminoalkylphosphoramidate, thionophosphoramidate, thionoalkylphosphonate, thionoalkylphosphotriester and boranophosphate, 2′-5′ linked analogues thereof, and adjacent pairs of nucleoside units having inverted polarity, which are linked 3′-5′ to 5′-3′ or 2′-5′ to 5′-2′. Various salts, mixed salts and free acid forms are also included. In some exemplary embodiments, the modified oligonucleotide is a single-stranded modified oligonucleotide. In some exemplary embodiments, the single-stranded modified oligonucleotide consists of 10 to 30, 10 to 35, 10 to 40, 10 to 45, 10 to 50, 10 to 60, 10 to 70, 10 to 80, 10 to 90, 10 to 100 or more than 100 linked nucleosides and has a gap segment. In some exemplary embodiments, the gap segment refers to one or more linked nucleic acids consisting of deoxynucleosides located at or near the center of a modified oligonucleotide, such as a single-stranded modified oligonucleotide. In some exemplary embodiments, the gap segment consists of 2 to 3, 2 to 4, 2 to 5, 2 to 6, 2 to 7, 2 to 8, 2 to 9, 2 to 10, 2 to 20, 2 to 30, 2 to 40 2 to 50, 10 to 20, 10 to 30, 10 to 40 or 10 to 50 linked deoxynucleosides. The 5′ wing segment corresponds to a nucleic acid (e.g., a nucleoside) linked from the 5′-end of the modified oligonucleotide to the nucleic acid preceding the first nucleic acid at the 5′-end of the gap segment. The 3′ wing segment corresponds to a nucleic acid (e.g., a nucleoside) linked from the last nucleic acid at the 3′ end of the gap segment to the last nucleic acid at the 3′ end of the modified oligonucleotide. The gap segment is located between the 5′ wing segment and the 3′ wing segment. In some exemplary embodiments, at least one nucleoside of the 5′ wing segment and/or at least one nucleoside of the 3′ wing segment includes a modified nucleoside. In some exemplary embodiments, all nucleoside linkages within the gap segment and all linkages connecting the gap segment to the 3′ wing segment and/or the 5′ wing segment are phosphorothioate linkages (*). In some exemplary embodiments, the internucleoside linkage connecting the remaining nucleosides of both the 5′ and 3′ wing segments is a phosphodiester linkage. In some exemplary embodiments, the nucleosides in the modified oligonucleotide are modified with a 2′ O-methyl group. Nucleosides in modified oligonucleosides may also be modified with any of the other modifications described herein. In some exemplary embodiments, the nucleobase sequence of the modified oligonucleotide consists of 10 to 30, 10 to 35, 10 to 40, 10 to 45, 10 to 50, 10 to 60, 10 to 70, 10 to 80, 10 to 90, 10 to 100 or more than 100 linked nucleosides and has a pharmaceutically acceptable salt thereof. Modified internucleoside linkages that do not contain a phosphorus atom therein have short-chain alkyl or cycloalkyl internucleoside linkages, mixed heteroatoms and alkyl or cycloalkyl internucleoside linkages, or one or more short-chain heteroatoms, or internucleoside linkages formed by heterocyclic internucleoside linkages. These include those with morpholino linkages (formed in part from the sugar moiety of the nucleoside); siloxane backbone; sulfide, sulfoxide and sulfone backbones; formacetyl and thioformacetyl backbones; methylene formacetyl and thioformacetyl backbones; alkene-containing backbones; sulphamate backbones; methylene imino and methylenehydrazino backbones; sulfonate and sulfonamide backbones; amide backbones; and others having mixed N, O, S and CH₂ component portions. Substituted sugar moieties include one of the following at the 2′ position, but the present disclosure is not limited thereto: H (deoxyribose); OH (ribose); F; O—, S- or N-alkyl; O—, S- or N-alkenyl; O—, S- or N-alkynyl; or O-alkyl-O-alkyl (where alkyl, alkenyl and alkynyl may be substituted or unsubstituted C1 to C10 alkyl or C2 to C10 alkenyl and alkynyl). Chemically or naturally modified oligonucleotides include, for example, at least one nucleotide modified at the 2′ position of the sugar, and most preferably, 2′-O-alkyl, 2′-O-alkyl-O-alkyl or 2′-fluoro-modified nucleotides or end caps. In other exemplary embodiments, RNA modifications include 2′-fluoro, 2′-amino and 2′ O-methyl modifications on the ribose of a pyrimidine, an abasic residue or an inverted base at the 3′ end of the RNA. Oligonucleotides that are useful in accordance with the present disclosure may include a single modified nucleoside. In other exemplary embodiments, the oligonucleotide may include at least 2 modified nucleosides, at least 3, at least 4, at least 5, at least 6, at least 7, at least 8, at least 9, at least 10, at least 15, at least 20 or more modified nucleosides up to the entire length of the oligonucleotide. Nucleosides or nucleobases, include the natural purine bases adenine (A) and guanine (G), and the pyrimidine bases thymine (T), cytosine (C) and uracil (U). Modified nucleosides include other synthetic and natural nucleobases, such as inosine, xanthine, hypoxanthine, nuvularine, isoguanicine, tubercidin, 2-(halo) adenine, 2-(alkyl) adenine, 2-(propyl) adenine, 2-(amino) adenine, 2-2-(aminopropyl) adenine, 2-(methylthio) N6 (aminoalkyl) adenine, (isopentenyl) adenine, 6-(alkyl) adenine, 6-(methyl) adenine, 7-(deaza) adenine, 8-(alkenyl) adenine, 8-(alkyl) adenine, 8-(alkynyl) adenine, 8-(amino) adenine, 8-(halo) adenine, 8-(hydroxy) adenine, 8-(thioalkyl) adenine, 8-(thiol) adenine, N6-(isopentyl) adenine, N6-(methyl) adenine, N6, N6-(dimethyl) adenine, 2-(alkyl) guanine, 2-(propyl) guanine, 6-(alkyl) guanine, 6-(methyl) guanine, 7-(alkyl) guanine, 7-(methyl) guanine, 7-(deaza) guanine, 8-(alkyl) guanine, 8-(alkenyl) guanine, 8-(alkynyl) guanine, 8-(amino) guanine, 8-(halo) guanine, 8-(hydroxyl) guanine, 8-(thioalkyl) guanine, 8-(thiol) guanine, N-(methyl) guanine, 2-(thio) cytosine, 3-(deaza)-5-(aza) cytosine, 3-(alkyl) cytosine, 3-(methyl) cytosine, 5-(alkyl) cytosine, 5-(alkynyl) cytosine, 5-(halo) cytosine, 5-(methyl) cytosine, 5-(propynyl) cytosine, 5-(propynyl) cytosine, 5-(trifluoromethyl) cytosine, 6-(azo) cytosine, N4-(acetyl) cytosine, 3-(3-amino-3 carboxypropyl) uracil, 2-(thio) uracil, 5-(methyl)-2-(thio) uracil, 5-(methylaminomethyl)-2-(thio) uracil, 4-(thio) uracil, 5-(methyl)-4-(thio) uracil, 5-(methylaminomethyl)-4-(thio) uracil, 5-(methyl)-2,4 (dithio) uracil, 5-(methylaminomethyl)-2,4 (dithio) uracil, 5-(2-aminopropyl) uracil, 5-(alkyl) uracil, 5-(alkynyl) uracil, 5-(allylamino) uracil, 5-(aminoallyl) uracil, 5-(aminoalkyl) uracil, 5-(guanidinium alkyl) uracil, 5-(1,3-diazole-1-alkyl) uracil, 5-(cyanoalkyl) uracil, 5-(dialkylaminoalkyl) uracil, 5-(dimethylaminoalkyl) uracil, 5-(halo) uracil, 5-(methoxy) uracil, uracil-5-oxyacetic acid, 5-(methoxycarbonylmethyl)-2-(thio) uracil, 5-(methoxycarbonyl-methyl) uracil, 5-(propynyl) uracil, 5-(propynyl) uracil, 5-(trifluoromethyl) uracil, 6-(azo) uracil, dihydrouracil, N3-(methyl) uracil, 5-uracil (i.e., pseudouracil), 2-(thio) pseudouracil, 4-(thio) pseudouracil, 2,4-(dithio) pseudouracil, 5-(alkyl) pseudouracil, 5-(methyl) pseudouracil, 5-(alkyl)-2-(thio) pseudouracil, 5-(methyl)-2-(thio) pseudouracil, 5-(alkyl)-4 (thio) pseudouracil, 5-(methyl)-4-(thio) pseudouracil, 5-(alkyl)-2,4-(dithio) pseudouracil, 5-(methyl)-2,4 (dithio) pseudouracil, 1-substituted pseudouracil, 1-substituted 2 (thio)-pseudouracil, 1-substituted 4-(thio) pseudouracil, 1-substituted 2,4-(dithio) pseudouracil, 1-(aminocarbonylethylenyl)-pseudouracil, 1-(aminocarbonylethylenyl)-2 (thio)-pseudouracil, 1-(aminocarbonylethylenyl)-4-(thio) pseudouracil, 1-(aminocarbonylethylenyl)-2,4-(dithio) pseudouracil, 1-(aminoalkylamino carbonylethylenyl)-pseudouracil, 1-(aminoalkylamino-carbonylethylenyl)-2 (thio)-pseudouracil, 1-(aminoalkylaminocarbonylethylenyl)-4 (thio) pseudouracil, 1-(aminoalkylaminocarbonylethylenyl)-2,4-(dithio) pseudouracil, 1,3-(diaza)-2-(oxo)-phenoxazine-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-penthiazin-1-yl, 7-substituted 1-(aza)-2-(thio)-3-(aza)-penthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidinium alkylhydroxy)-1,3-(diaza)-2-(oxo)-phenoxazin-1-yl, 7-(guanidinium alkylhydroxy)-1-(aza)-2-(thio)-3-(aza)-phenoxazin-1-yl, 7-(guanidinium alkyl-hydroxy)-1,3-(diaza)-2-(oxo)-phenthiazin-1-yl, 7-(guanidinium alkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularin, tubersidin, isoguanisine, inosynyl, 2-aza-inosynyl, 7-deaza-inosynyl, nitroimidazolyl, nitropyrazolyl, nitrobenzimidazolyl, nitroindazolyl, aminoindolyl, pyrrolopyrimidinyl, 3-(methyl) isocarbostyrylyl, 5-(methyl) isocarbostyrylyl, 3-(methyl)-7-(propynyl) isocarbostyrylyl, 7-(aza) indolyl, 6-(methyl)-7-(aza) indolyl, imidazopyridinyl, 9-(methyl)-imidazopyridinyl, pyrrolopyrizinyl, isocarbostyryl, 7-(propynyl) isocarbostyryl, propynyl-7-(aza) indolyl, 2,4,5-(trimethyl)phenyl, 4-(methyl) indolyl, 4,6-(dimethyl) indolyl, phenyl, naphthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo) thymine, 2-pyridinone, 5 nitroindole, 3 nitropyrrole, 6-(aza)pyrimidine, 2-(amino) purine, 2,6-(diamino) purine, 5-substituted pyrimidine, N2-substituted purine, N6-substituted purine, 06-substituted purine, substituted 1,2,4-triazole, pyrrolo-pyrimidin-2-one-3-yl, 6-phenyl-pyrrolo-pyrimidin-2-one-3-yl, para-substituted-6-phenyl-pyrrolo-pyrimidin-2-one-3-yl, ortho-substituted-6-phenyl-pyrrolo-pyrimidin-2-one-3-yl, bis-ortho-substituted-6-phenyl-pyrrolo-pyrimidine-2-one-3-yl, para-(aminoalkylhydroxy)-6-phenyl-pyrrolo-pyrimidin-2-one-3-yl, ortho-(aminoalkylhydroxy)-6-phenyl-pyrrolo-pyrimidin-2-one-3-yl, bis-ortho-(aminoalkylhydroxy)-6-phenyl-pyrrolo-pyrimidin-2-one-3-yl, pyridopyrimidin-3-yl, 2-oxo-7-amino-pyri-dopyrimidin-3-yl, 2-oxo-pyridopyrimidin-3-yl, or any O-alkylated or N-alkylated derivatives thereof. The antisense oligonucleotides of the present disclosure may be chimeric oligonucleotides. The chimeric antisense compounds of the present disclosure may be formed of a complex structure of two or more oligonucleotides, modified oligonucleotides, oligonucleotides and/or oligonucleotide mimetics described above. These compounds have also been referred to in the art as hybrids or mixed backbones or chimeras or gapmers. In particular, the gapmer is an oligonucleotide having at least 3 distinct segments, where two of the segments are similar, that is, it includes one or more backbone modifications, and surrounds a region that is distinct (i.e., not involved in the backbone modification). Oligonucleotides may include molecular species at one or both ends, that is, at the 3′ and/or 5′ ends. As used herein, the molecular species refers to any compound that is not a naturally occurring or non-naturally occurring nucleotide. The molecular species includes spacers, lipids, sterols, lipid moieties such as cholesterol moieties, cholic acid, thioethers such as hexyl-S-tritylthiol, thiocholesterol, aliphatic chains such as dodecanediol or undecyl moieties, phospholipids such as di-hexadecyl-rac-glycerol or triethylammonium 1,2-di-O-hexadecyl-rac-glycero-3-H-phosphonate, polyamine or polyethylene glycol chains, or adamantane acetic acid, palmityl moiety, octadecylamine or hexylamino-carbonyl-oxycholesterol moiety, stearyl, C16 alkyl chain, bile acid, cholic acid, taurocholic acid, deoxycholate, oleyl lithocholic acid, oleoyl cholenic acid, glycolipids, phospholipids, sphingolipids, isoprenoids such as steroids, vitamins such as vitamin E, saturated fatty acids, unsaturated fatty acids, fatty acid esters such as triglycerides, pyrene, porphyrin, texaphyrin, adamantane, acridine, biotin, coumarin, fluorescein, rhodamine, Texas-red, digoxigenin, dimethoxytrityl, t-butyldimethylsilyl, t-butyldiphenylsilyl, cyanine dyes (e.g. Cy3 or Cy576), Hoekst 33258 dye, psoralen or ibuprofen, but the present disclosure is not limited thereto.

Molecular species may be attached at various positions on the oligonucleotide.

As described above, the molecular species may be linked to the 2′-end, 3′-end or 5′-end of the oligonucleotide, where it also serves to improve the stability of the oligomers toward 3′- or 5′-exonucleases.

Alternatively, it may be linked to an internal nucleotide or to a nucleotide on a branch. The molecular species may be attached to the 2′-position of the nucleotide. The molecular species may also be linked to heterocyclic bases of nucleotides. The molecular species may be linked to oligonucleotides by linker moieties. Optionally, the linker moiety is a non-nucleotide linker moiety. Non-nucleotide linkers are, for example, abasic moieties (disspacers), oligoethylene glycols such as triethylene glycol or hexaethylene glycol, or alkane-diols such as butanediol. The spacer units are preferably linked by phosphodiester, phosphorodithioate or phosphorothioate bonds. The linker unit may be present alone in the molecule or may be introduced multiple times, for example via phosphodiester, phosphorothioate, methylphosphonate or amide linkages. The oligonucleotides of the present disclosure may also contain non-nucleotide linkers (apart from the linkers connecting the nucleotides to the molecular species), particularly, abasic linkers (disspacers), triethylene glycol units or hexaethylene glycol units. Additionally preferred linkers are alkylamino linkers, such as C3, C6 or C12 amino linkers, and also alkylthiol linkers, such as C3 or C6 thiol linkers.

From another perspective, the present disclosure relates to a pharmaceutical composition for preventing or treating COVID-19, including the oligonucleotide or a pharmaceutically acceptable salt of the oligonucleotide as an active ingredient.

In the present disclosure, the pharmaceutical composition may further include a carrier in order to increase intracellular delivery efficiency of the oligonucleotide or a pharmaceutically acceptable salt of the oligonucleotide.

Examples of carriers for delivering the oligonucleotide or a pharmaceutically acceptable salt of the oligonucleotide into cells include viral vectors, non-viral vectors, liposomes, cationic polymers, micelles, emulsions, lipid nanoparticles (solid lipids). nanoparticles), exosomes and the like. The viral vectors have the advantage of high delivery efficiency and long duration. The viral vectors include retroviral vectors, adenoviral vectors, vaccinia virus vectors, adeno-associated viral vectors, cancer cell lytic viral vectors and the like. The non-viral vectors may include plasmids. In addition, various dosage forms such as liposomes, cationic polymers, micelles, emulsions, solid lipid nanoparticles and the like may be used. Examples of cationic polymers for nucleic acid delivery include natural polymers such as chitosan, atelocollagen and cationic polypeptide, as well as synthetic polymers such as poly(L-lysin), linear or branched polyethylene imine (PEI), cyclodextrin-based polycation, dendrimers and the like.

The lipid nanoparticles form nucleic acid-lipid particles together with the oligonucleotide or a pharmaceutically acceptable salt of the oligonucleotide. Nucleic acid-lipid particles usually include cationic lipids, non-cationic lipids, sterols and lipids that prevent aggregation of the particles (e.g., PEG-lipid conjugates). Since nucleic acid-lipid particles exhibit a prolonged circulating lifespan after i.v. injection and accumulate at distal sites (e.g., sites that are physically distant from the site of administration), they are extremely useful for whole body applications. In addition, when nucleic acids are present in the nucleic acid-lipid particles of the present disclosure, they are resistant to degradation by nucleic acid degrading enzymes in aqueous solutions. Nucleic acid-lipid particles and the preparation methods thereof are described, for example, in U.S. Pat. Nos. 5,976,567; 5,981,501; 6,534,484; 6,586,410; 6,815,432; and WO 96/40964.

Nucleic acid-lipid particles may also include one or more additional lipids and/or other components, such as cholesterol. Other lipids may be included in the liposome composition for various purposes, such as to prevent lipid oxidation or to attach ligands on the liposome surface. Any number of lipids may be present, including amphipathic, neutral, cationic and anionic lipids. These lipids may be used alone or in combination.

Additional components that may be present in the nucleic acid-lipid particles include bilayer stabilizing components such as polyamide oligomers (e.g., refer to U.S. Pat. No. 6,320,017), peptides, proteins, detergents and lipid-derivatives, such as PEG and ceramides conjugated to phosphatidylethanolamine (refer to U.S. Pat. No. 5,885,613). The nucleic acid-lipid particles may include one or more second amino lipids or cationic lipids, neutral lipids, sterols and lipids selected to reduce the aggregation of lipid particles during formation, which may contribute to the structural stabilization of particles preventing charge-induced aggregation during formation.

The nucleic acid-lipid particles may be produced through an extrusion method or an in-line mixing method. The extrusion method (also called a batch process) is a method of first making empty liposomes (e.g., without nucleic acids) and then adding nucleic acids to the empty liposomes, and it is described in U.S. Pat. Nos. 5,008,050; 4,927,637; 4,737,323; Biochim Biophys Acta. 1979 Oct. 19; 557 (1): 9-23; Biochim Biophys Acta. 1980 Oct. 2; 601 (3): 559-7; Biochim Biophys Acta. 1986 Jun. 13; 858 (1): 161-8; and Biochim. Biophys. Acta 1985 812, 55-65, and the full texts thereof may be referenced herein.

The in-line mixing method is a method in which lipids and nucleic acids are added side by side to a mixing chamber. The mixing chamber may be a simple T-connector or any other mixing chamber known in the art. These methods are disclosed in U.S. Pat. Nos. 6,534,018 and 6,855,277; US Patent Application Publication No. 2007-0042031 and Pharmaceuticals Research, Vol. 22, No. 3, March 2005, pp. 362-372, and the full texts may be referenced herein.

According to one embodiment, nucleic acid-lipid particles may be synthesized by using a fatty substance (lipidoid) ND98 (MW 1487), cholesterol and PEG-ceramide C16. Lipid-siRNA nanoparticles generally form spontaneously upon mixing. Depending on the desired particle size distribution, the resulting nanoparticle mixture may be extruded through a polycarbonate membrane (e.g., 100 nm cut-off) by using a thermobarrel extruder, such as a Lipex Extruder (Northern Lipid, Inc). In some cases, the extrusion step may be omitted. Ethanol removal and simultaneous buffer exchange may be accomplished by dialysis or tangential flow filtration. The buffer may be replaced with phosphate buffered salts (PBS) at about pH 7, for example, about pH 6.9, about pH 7.0, about pH 7.1, about pH 7.2, about pH 7.3 or about pH 7.4.

The oligonucleotide or a pharmaceutically acceptable salt of the oligonucleotide may be introduced into cells in vivo or in vitro in the form of a complex with a carrier.

The dosage form of the present disclosure may be prepared by any method known in the art.

In the present disclosure, the term “prevention” refers to all actions that inhibit or delay the occurrence, spread and recurrence of COVID-19 virus infection by administering the composition according to the present disclosure.

The term “treatment” used in the present disclosure refers to all actions that ameliorate or beneficially change the symptoms of COVID-19 virus infection and complications resulting therefrom by administering the composition according to the present disclosure. Those skilled in the art to which the present disclosure pertains may refer to the materials presented by the Korean Medical Association and the like to understand the exact criteria for diseases for which the composition of the present disclosure is effective and to determine the degree of amelioration, improvement and treatment.

The present disclosure may provide a pharmaceutical composition including the oligonucleotide or a pharmaceutically acceptable salt of the oligonucleotide in a therapeutically effective amount, and in the present disclosure, the term “therapeutically effective amount” used in combination with the active ingredient refers to an amount that is effective in preventing or treating COVID-19 virus infection, and the therapeutically effective amount of the composition of the present disclosure may vary depending on various factors, such as administration method, target site, the patient's condition and the like. Therefore, when used in the human body, the dosage must be determined as an appropriate amount by considering both safety and efficiency. It is also possible to estimate the amount used in humans from the effective amount determined through animal testing. These considerations in determining an effective amount are described in, for example, Hardman and Limbird, eds., Goodman and Gilman's The Pharmacological Basis of Therapeutics, 10^{th} ed. (2001), Pergamon Press; and E. W. Martin ed., Remington's Pharmaceutical Sciences, 18^{th} ed. (1990), Mack Publishing Co.

The composition of the present disclosure is administered in a pharmaceutically effective amount. The term “pharmaceutically effective amount” used herein refers to an amount that is sufficient to treat the disease at a reasonable benefit/risk ratio applicable for medical treatment and an amount that does not cause side effects. The level of an effective dosage may be determined by parameters including the health status of a patient, the severity of COVID-19 virus infection, the activity of a drug, sensitivity to a drug, an administration method, administration time, an administration route and a release rate, the duration of treatment, formulated or co-used drugs and other parameters well known in medical fields. The composition of the present disclosure may be administered as an individual therapeutic agent or in combination with other therapeutic agents. It may be administered sequentially or simultaneously with a conventional therapeutic agent or administered in a single- or multiple-dose regime. In consideration of all of the above parameters, it is important to administer such a dose to obtain a maximum effect with a minimal amount without a side effect, and the dose may be easily determined by those skilled in the art.

The pharmaceutical composition of the present disclosure may include carriers, diluents, excipients or a combination of two or more thereof commonly used in biological formulations. The term “pharmaceutically acceptable” as used herein means that the composition is free of toxicity to cells or humans exposed to the composition. The carrier is not particularly limited as long as it is suitable for the delivery of the composition to the living body. For example, compounds, saline solutions, sterile water, Ringer's solution, buffered saline, dextrose solution, maltodextrin solution, glycerol and ethanol disclosed in Merck Index, 13^{th} ed., Merck & Co. Inc. and one or more ingredients thereof may be mixed and used. If necessary, conventional additives such as antioxidants, buffers and bacteriostatic agents may be added. In addition, the composition may also be prepared into dosage form for injection such as aqueous solution, suspension, or emulsion, tablet, capsule, powder or pill by additionally including diluents, dispersant, surfactant, binder and lubricant. Furthermore, the composition may be formulated into a desirable form depending on each targeting disease or ingredients thereof, by using the method disclosed in Remington's Pharmaceutical Science (Mack Publishing Company, Easton Pa., 18^{th}, 1990).

In an embodiment, the pharmaceutical composition may be one or more formulations selected from the group consisting of oral formulations, external preparations, suppositories, sterile injectable solutions and sprays, and more preferably, oral formations or injectable formulations.

As used herein, the term “administration” means providing a predetermined substance to a subject or a patient by any appropriate method and may be administered orally or parenterally (e.g., by applying in injectable formulations intravenously, subcutaneously, intraperitoneally or topically). The dosage may vary depending on the patient's body weight, age, gender, health condition, diet, administration time, administration method, excretion rate, the severity of the disease and the like. The liquid formulations for oral administration of the composition of the present disclosure include suspensions, oral liquids, emulsions, syrups and the like. In addition to water and liquid paraffin which are simple diluents that are commonly used, various excipients such as wetting agents, sweeteners, flavors, preservatives and the like may be included. Formulations for parenteral administration include sterile aqueous solutions, non-aqueous solvents, suspensions, emulsions, freeze-dried formulations, suppositories and the like. The pharmaceutical composition of the present disclosure may be administered by any device which is capable of moving the active substance to target cells. The preferred administration method and formulations include intravenous, subcutaneous, intradermal, intramuscular, drip injections and the like. The injectable solution may be prepared by using an aqueous solvent such as a physiological saline solution and Ringer's solution, and a non-aqueous solvent such as a vegetable oil, a higher fatty acid ester (e.g., ethyl oleate), an alcohol (e.g., ethanol, benzyl alcohol, propylene glycol, glycerin, etc.) and may include pharmaceutical carriers such as stabilizer to prevent deterioration (e.g., ascorbic acid, sodium hydrogen sulfite, sodium pyrosulfite, BHA, tocopherol, EDTA, etc.), an emulsifier, a buffer for pH control, preservatives for the inhibition of microbial growth (e.g., phenylmercuric nitrate, thimerosal, benzalkonium chloride, phenol, cresol, benzyl alcohol, etc.) and the like.

As used herein, the term “subject” means all animals who have developed the COVID-19 virus infection or are capable of developing the COVID-19 virus infection, including human, a monkey, a cow, a horse, sheep, a pig, a chicken, a turkey, a quail, a cat, a dog, a mouse, a bat, a camel, a rat, a rabbit or a guinea pig, and the term “specimen” may be droplets, sputum, whole blood, plasma, serum, urine or saliva isolated therefrom.

The pharmaceutical compositions described herein may be prepared by any method known in the art of pharmacology or as discussed later in the text. Generally, these methods for tableting involve the steps of associating the active ingredient with excipients and/or one or more other auxiliary ingredients, and subsequently, if necessary or desired, the step of forming and/or packaging the product into desired single- or multi-dose units is included.

The pharmaceutical composition of the present disclosure may be prepared, packaged and/or sold unpackaged as a single unit dose and/or multiple single-unit doses. As used herein, the term “unit dose” is a discrete amount of a pharmaceutical composition including a predetermined amount of an active ingredient. The amount of active ingredient is generally equal to the dosage of active ingredient administered to the subject and/or a convenient fraction of such dosage such as, for example, one-half or one-third of the dosage.

The relative amounts of the active ingredient, pharmaceutically acceptable excipients and/or any additional ingredients in the pharmaceutical composition of the present disclosure will vary depending on the identity, size and/or disorder of the subject being treated and the route by which the composition is administered. By way of example, the composition may include 0.1% to 100% (w/w) of the active ingredient.

As used herein, pharmaceutically acceptable excipients include any and all of solvent, dispersion medium, diluent, or other liquid vehicle, dispersion or suspension aid, surface active agent, tonicity agent, thickener or emulsifier, preservative, solid binder, lubricant and the like that are suitable for the purpose of a particular dosage form. Remington's literature [The Science and Practice of Pharmacy, 21^{st} Edition, A. R. Gennaro, (Lippincott, Williams & Wilkins, Baltimore, MD, 2006) describes various excipients used in the formulation of pharmaceutical composition and techniques known for their preparation. A use of any conventional carrier medium is considered to be within the scope of the present disclosure, except that it is incompatible with a substance or its derivative, for example, by providing any unwanted biological effect or otherwise interacting with any other component of pharmaceutical compositions in a harmful manner. Pharmaceutically acceptable excipients are at least 95%, 96%, 97%, 98%, 99% or 100% pure.

In some exemplary embodiments, the excipients are approved for human and veterinary use. In some exemplary embodiments, the excipients are approved by the US Food and Drug Administration. In some exemplary embodiments, the excipients are pharmaceutical grade. In some exemplary embodiments, the excipients meet the standards of the United States Pharmacopoeia (USP), European Pharmacopeia (EP), British Pharmacopoeia and/or International Pharmacopoeia (Ph. Int.).

Pharmaceutically acceptable excipients used in the preparation of pharmaceutical compositions include inert diluents, dispersants and/or granulizers, surface active agents and/or emulsifiers, disintegrants, binders, preservatives, buffers, lubricants, and/or oils, but the present disclosure is not limited thereto.

Such excipients may optionally be included in the formulations of the present disclosure. Excipients such as cocoa butter and suppository wax, colorants, coatings, sweeteners, flavors and perfumes may be present in the composition at the discretion of the formulator.

Exemplary diluents include calcium carbonate, sodium carbonate, calcium phosphate, dicalcium phosphate, calcium sulfate, calcium hydrogen phosphate, sodium lactose phosphate, sucrose, cellulose, microcrystalline cellulose, kaolin, mannitol, sorbitol, inositol, sodium chloride, dried starch, corn starch, powdered sugar and combinations thereof, but the present disclosure is not limited thereto.

Exemplary granulating and/or dispersing agents include potato starch, corn starch, tapioca starch, sodium starch glycolate, clay, alginic acid, guar gum, citrus pulp, agar, bentonite, cellulose and wood products, natural sponges, cation-exchange resins, calcium carbonate, silicate, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (veegum), sodium lauryl sulfate, quaternary ammonium compounds and the combinations thereof, but the present disclosure is not limited thereto.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax and lecithin), colloidal clay (e.g., bentonite [aluminum silicate] and veegum [magnesium aluminum silicate]), long-chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol); carbomers (e.g., carboxy polymethylene, polyacrylic acid, acrylic acid polymers, and carboxyvinyl polymers), carrageenan, cellulose derivatives (e.g., carboxymethyl cellulose sodium, powdered cellulose, hydroxymethyl cellulose, hydroxypropyl cellulose, hydroxypropyl methyl cellulose, methyl cellulose), sorbitan fatty acid esters (e.g., polyoxyethylene sorbitan monolaurate [Tween 20], polyoxyethylene sorbitan [Tween 60], polyoxyethylene sorbitan monooleate [Tween 80], sorbitan monopalmitate [span 40], sorbitan monostearate [span 60], sorbitan tristearate [span 65], glyceryl monooleate, sorbitan monooleate [span 80]), polyoxyethylene esters (e.g., polyoxyethylene monostearate [myrz 45], polyoxyethylene hydrogenated castor oil, polyethoxylated castor oil, polyoxymethylene stearate and solutol), sucrose fatty acid esters, polyethylene glycol fatty acid esters (e.g., cremophor), polyoxyethylene ethers (e.g., polyoxyethylene lauryl ether [Breeze 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium and/or combinations thereof, but the present disclosure is not limited thereto.

Exemplary binders include starches (e.g., corn starch and starch paste); gelatin; sugars (e.g., sucrose, glucose, dextrose, dextrin, molasses, lactose, lactitol, mannitol); natural and synthetic gums (e.g., acacia, sodium alginate, extract of Irish moss, panwar gum, shatty gum, mucilage of isapol husks, carboxymethyl cellulose, methyl cellulose, ethyl cellulose, hydroxyethyl cellulose, hydroxypropyl cellulose, hydroxypropyl methyl cellulose, microcrystalline cellulose, cellulose acetate, poly(vinyl-pyrrolidone), magnesium aluminum silicate (veegum), and larch arabinogalactan); alginate; polyethylene oxide; polyethylene glycol; inorganic calcium salts; silicic acid; polymethacrylate; wax; water; alcohol; and combinations thereof, but the present disclosure is not limited thereto.

Exemplary preservatives may include antioxidants, chelating agents, antimicrobial preservatives, antifungal preservatives, alcohol preservatives, acidic preservatives, and other preservatives. Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite, but the present disclosure is not limited thereto. Exemplary chelating agents include ethylene diamine tetraacetic acid (EDTA), citric acid monohydrate, disodium edetate, dipotassium edetate, edetic acid, fumaric acid, malic acid, phosphoric acid, sodium edetate, tartaric acid and trisodium edetate. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol and thimerosal, but the present disclosure is not limited thereto. Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate and sorbic acid, but the present disclosure is not limited thereto. Exemplary alcohol preservatives include ethanol, polyethylene glycol, phenol, phenolic compounds, bisphenol, chlorobutanol, hydroxybenzoate and phenylethyl alcohol, but the present disclosure is not limited thereto. Exemplary acidic preservatives include vitamin A, vitamin C, vitamin E, beta-carotene, citric acid, acetic acid, dehydroacetic acid, ascorbic acid, sorbic acid and phytic acid, but the present disclosure is not limited thereto. Other preservatives include tocopherol, tocopherol acetate, deteroxime mesylate, cetrimide, butylated hydroxyanisole (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glidant Plus, Fenonib, methylparaben, Germall 115, Germaben II, Neolone, Kathon and Euxyl, but the present disclosure is not limited thereto. In certain exemplary embodiments, the preservative is an anti-oxidant. In another embodiment, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol and combinations thereof, but the present disclosure is not limited thereto.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate and combinations thereof, but the present disclosure is not limited thereto.

Exemplary oils include almond, apricot kernel, avocado, babassu palm, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, pumpkin, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut and wheat germ oil, but the present disclosure is not limited thereto. Exemplary oils include butyl stearate, caprylic triglyceride, capric triglyceride, cyclomethicone, diethyl sebacate, dimethicone 360, isopropyl myristate, mineral oil, octyldodecanol, oleyl alcohol, silicone oil and combinations thereof, but the present disclosure is not limited thereto.

Liquid dosage forms for oral and parenteral administration include pharmaceutically acceptable emulsions, microemulsions, solutions, suspensions, syrups and elixirs, but the present disclosure is not limited thereto. In addition to active ingredients, the liquid dosage forms may also include inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (particularly, cotton seed, groundnut, corn, germ, olive, castor, and sesame oils), glycerol, tetrahydrofurfuryl alcohol, polyethylene glycols and fatty acid esters of sorbitan and mixtures thereof. Besides inert diluents, oral compositions may include adjuvants such as wetting agents, emulsifying and suspending agents, sweetening agents, flavoring agents and perfuming agents. In certain exemplary embodiments for parenteral administration, the chimeric compounds of the present disclosure are mixed with solubilizing agents such as Cremophor, alcohols, oils, modified oils, glycols, polysorbates, cyclodextrins, polymers and combinations thereof.

Injectable preparations, for example, sterile injectable aqueous or oleaginous suspensions may be formulated according to the known art using dispersing agents, wetting agents and/or suspending agents. Sterile injectable preparations may be sterile injectable solutions, suspensions, or emulsions in nontoxic parenterally acceptable diluents or solvents such as 1,3-butanediol. Among the acceptable vehicles and solvents, water, Ringer's solution, U.S.P., and isotonic sodium chloride solution may be employed. In addition, sterile and fixed oils are conventionally employed as a solvent or suspending medium. For this purpose, any bland fixed oil, including synthetic mono- or di-glycerides, may be employed. In addition, fatty acids such as oleic acid are used in the preparation of injectables.

Injectable formulations may be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This is accomplished by the use of a liquid suspension of crystalline or amorphous material with poor water solubility. The rate of absorption of the drug then depends upon the rate of dissolution which, in turn, may depend upon crystal size and crystalline form. Alternatively, delayed absorption of a parenterally administered drug is accomplished by dissolving or suspending the drug in an oil vehicle.

Formulations for topical administration include liquid and/or semi-liquid preparations such as liniments, lotions, oil in water and/or water in oil emulsions such as creams, ointments and/or pastes, and/or solutions and/or suspensions, but the present disclosure is not limited thereto. Although the concentration of the active ingredient may be as high as the solubility limit of the active ingredient in the solvent, topically-administrable formulations may include, for example, from about 1% to about 10% (w/w) of the active ingredient. Formulations for topical administration may further include one or more of the additional ingredients described herein.

The pharmaceutical composition of the present disclosure may be prepared, packaged and sold as a formulation for pulmonary administration via the oral cavity. Such formulations may also include dry particles including the active ingredient and having a diameter within a range of about 0.5 to about 7 nanometers or about 1 to about 6 nanometers. Such compositions are conveniently in the form of a dry powder for administration using a device including a dry powder reservoir into which a stream of propellant may be directed to disperse the powder, and for administration using a device including the active ingredient dissolved and/or suspended in a low-boiling propellant in a self-propelled solvent/powder dispensing vessel such as a sealed vessel. Such powders include particles in which at least 98% of the particles by weight have a diameter greater than 0.5 nanometers and at least 95% of the particles by number have a diameter less than 7 nanometers. Alternatively, at least 95% of the particles by weight have a diameter greater than 1 nanometer, and at least 90% of the particles by number have a diameter less than 6 nanometers. Dry powder compositions may also include a solid fine powder diluent such as a sugar and are conveniently provided in unit dose form.

The low boiling propellant generally includes a liquid propellant having a boiling point below 65° F. at atmospheric pressure. In general, the propellant may constitute 50 to 99.9% (w/w) of the composition, and the active ingredient may constitute 0.1 to 20% (w/w) of the composition. The propellant may further include additional components such as a liquid non-ionic and/or solid anionic surfactant and/or a solid diluent (which may be of the same grade of particle size as the particles including the active ingredient).

The pharmaceutical composition of the present disclosure formulated for pulmonary delivery may provide the active ingredient in droplet form of a solution and/or a suspension. Such formulations may be prepared, packaged and/or sold as aqueous and/or dilute alcoholic solutions and/or suspensions, optionally sterilization containing the active ingredient, and may conveniently be administered using any nebulization and/or atomization device. Such formulations may further include one or more additional ingredients including, but not limited to, flavoring agents such as saccharin sodium, volatile oils, buffering agents, surface active agents and/or preservatives such as methylhydroxybenzoate. Droplets provided by this route of administration may have an average diameter within a range of about 0.1 to about 200 nanometers.

Formulations described herein useful for pulmonary delivery are useful for intranasal delivery of the pharmaceutical compositions of the present disclosure. Another formulation for intranasal administration is a coarse powder including the active ingredient and having an average particle from about 0.2 to 500 micrometers. Such a formulation is administered in the manner in which snuff is taken, that is, by rapid inhalation through the nasal passage from a container of the powder held close to the nose.

Formulations for nasal administration may, for example, include from about 0.1% (w/w) to 100% (w/w) of the active ingredient, and may also include one or more additional ingredients described herein. The pharmaceutical composition of the present disclosure may be prepared, packaged and sold as a formulation for oral administration. Such formulations may be in the form of, for example, tablets and/or lozenges prepared in a conventional manner, and may include, for example, 0.1 to 20% (w/w) of the active ingredient, the balance including an orally dissolvable and/or degradable composition and, optionally, one or more of the additional ingredients described herein. Alternatively, formulations for oral administration may include powders and/or aerosolized and/or atomized solutions and/or suspensions containing the active ingredients. When dispersed, such powdered, aerosolized and/or atomized formulations may have a droplet size and/or average particle size within a range of about 0.1 to about 200 nanometers, and may further include one or more additional ingredients described herein.

Hereinafter, the present disclosure will be described in more detail through examples. These examples are only for describing the present disclosure in more detail, and it will be apparent to those skilled in the art that the scope of the present disclosure is not limited to these examples.

In the examples, +A, +T, +G and +C represent LNA, and A, T, C and G represent DNA.

### Example 1. Literature Search and Effectiveness Verification of Antisense Oligonucleotide Targeting TIS-L

It is predicted that an antisense oligonucleotide targeting TIS-L effectively inhibits the viral replication of SARS-COV-2, and thus, in order to verify this, a literature search was conducted for antisense oligonucleotides targeting TIS-L. As a result, it was confirmed in the literature by Neuman (Journal of Virology 79.15 (2005): 9665-9676) that an antisense oligonucleotide targeting TIS-L was constructed to inhibit coronavirus, and it was determined if the corresponding antisense oligonucleotide effectively inhibits the viral replication of SARS-COV-2. The antisense oligonucleotide of Vora (Proceedings of the National Academy of Sciences 119.9 (2022): e2117198119), which was confirmed to have an inhibitory effect on SARS-COV-2, was used as a positive control.
Neuman sequence (SEQ ID NO: 1): +T+A+AAGTTCGTTTAGAGAA+C+A+G [Manufacturer: IDT]
Vora sequence (SEQ ID NO: 2): +C+C+TGGGAAGGTAAAACCTTT+A+A+T [Manufacturer: BIONEER]

In order to evaluate the antiviral efficacy of the present disclosure, a biosafety level 3 facility (Biosafety Level 3, BSL3) which is capable of performing experiments that are related to highly pathogenic viral pathogens at Korea University College of Medicine was used.

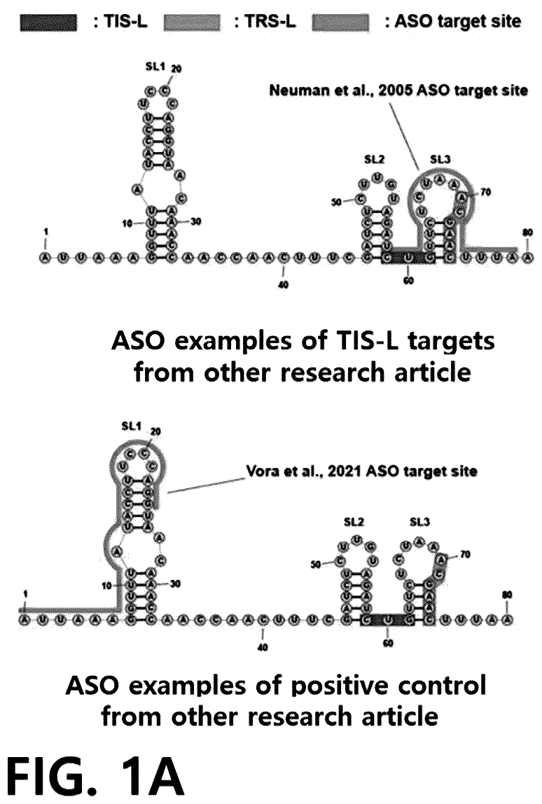

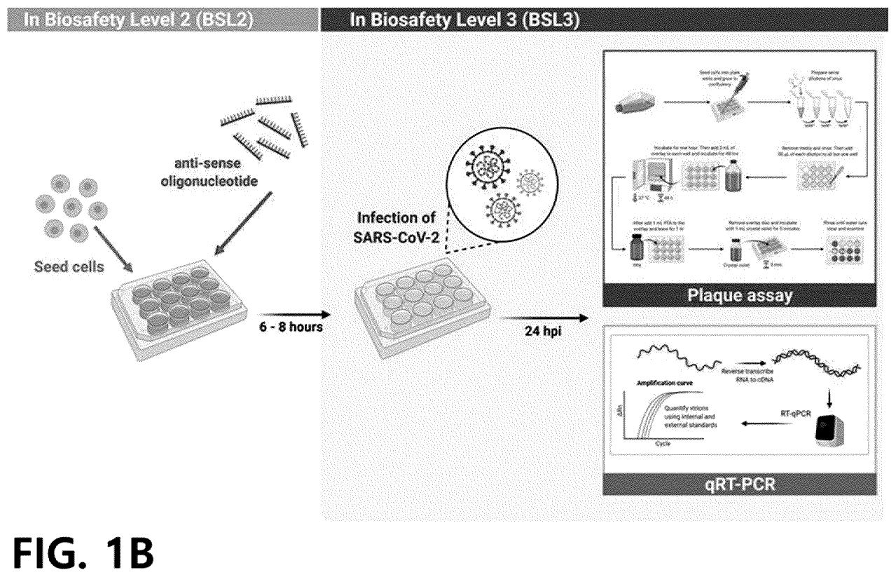

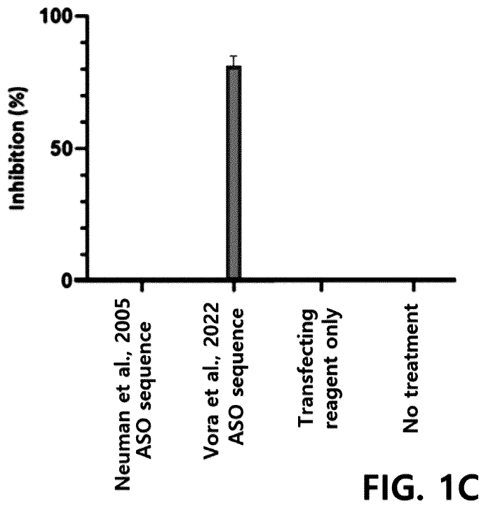

The inventors of the present disclosure established a plaque formation test (plaque assay) and qRT-PCR-based antiviral treatment effectiveness evaluation method for the COVID-19 virus based on the Vero cell line, and conducted an antiviral efficacy evaluation experiment based thereon. Specifically, as shown in FIG. 1B, the Vero cell line was treated with an antisense oligonucleotide (750 nM) and Lipofectamine 3000 (Thermo Fisher), which is a transfecting reagent, according to the manufacturer's instructions, and after infecting with SARS-COV-2 virus 6 to 8 hours later, 24 hours had elapsed, and then, qRT-PCR and plaque assay were performed to observe viral replication. In order to perform qRT-PCR, the supernatant was obtained, and RNA was extracted by using the Maxwell RSV Viral Total Nucleic Acid Purification kit (Promega), and afterwards, qRT-PCR was performed by using the primers below.
Forward: (5′-GTGARATGGTCATGTTGTGGCGG)
Reverse: (5′-CARATGTTAAASACACTATTAGCATA)
Probe: (5′-FAMCAGTGGAACCTCATCAGGAGATGC-BHQI)

For plaque assay, after infecting Vero cells that were laid as a monolayer on a 6-well plate with a diluted supernatant, it was covered with DMEM-F12 (Sigma-Aldrich) medium supplemented with 2% agarose, and after 72 hours, it was stained with crystal violet (Georgia Chemicals Inc.). The qRT-PCR and plaque assay experimental methods were performed as described in Bae & Lee (Biomolecules & Therapeutics 2021; 29 (3): 268-272).

The used Vero cell line (KCLB Cat #10081) was purchased from the Korean Cell Line Bank, and the SARS-COV-2 (BetaCoV/Korea/KCDC03/2020, NCCP no. 43326) virus was purchased from the Korea Disease Control and Prevention Agency's National Culture Collection for Pathogens (NCCP). The distributed virus was propagated in the Vero cell line.

As a result, as shown in FIG. 1C, the antisense oligonucleotide disclosed in Vora exerted a viral replication inhibitory effect on SARS-COV-2, whereas the antisense oligonucleotide targeting TIS-L as disclosed by Neuman had a relatively insignificant inhibitory effect on the viral replication of SARS-COV-2.

### Example 2. Construction and Effectiveness Verification of 20 Mer Antisense Oligonucleotides Targeting TIS-L

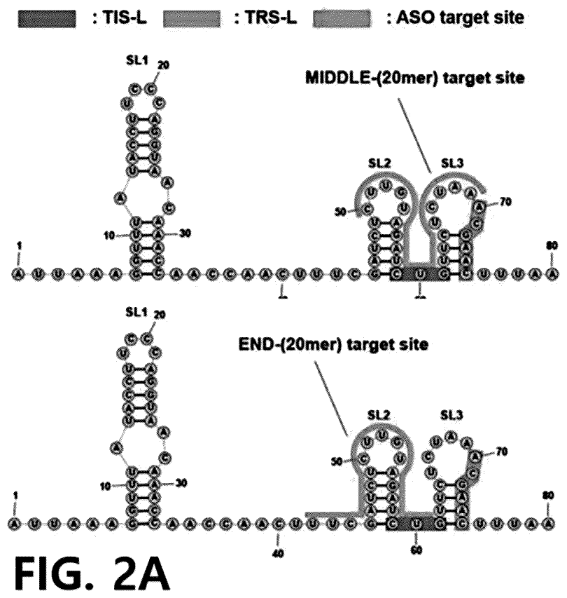

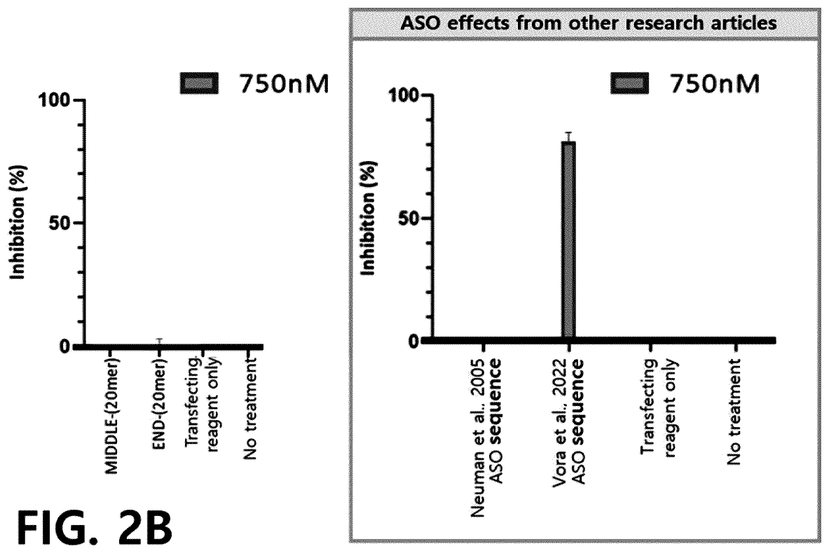

Although the antisense oligonucleotide targeting TIS-L as disclosed in Neuman's literature exhibited a weak SARS-COV-2 inhibitory effect, in the present disclosure, two 20 mer antisense oligonucleotides targeting TIS-L were additionally constructed (FIG. 2A), and the efficacy as an antiviral therapeutic agent was evaluated by conducting an inhibition experiment on the viral replication of SARS-COV-2 by using the same method as Example 1.
MIDDLE-(20 mer) sequence (SEQ ID NO: 3): +T+T+AGAGAACAGATCTAC+A+A+G [Manufacturer: IDT]
END-(20 mer) sequence (SEQ ID NO: 4): +C+A+GATCTACAAGAGATC+G+A+A [Manufacturer: IDT]

As a result, as shown in FIG. 2B, it was confirmed that both of the END-(20 mer) antisense oligonucleotide with a complementary binding site to TIS-L located at the 5′ end and the MIDDLE-(20 mer) antisense oligonucleotide with a complementary binding site to TIS-L located in the middle did not effectively inhibit the viral replication of SARS-COV-2.

### Example 3. Construction and Effectiveness Verification of 24 Mer Antisense Oligonucleotides Targeting TIS-L

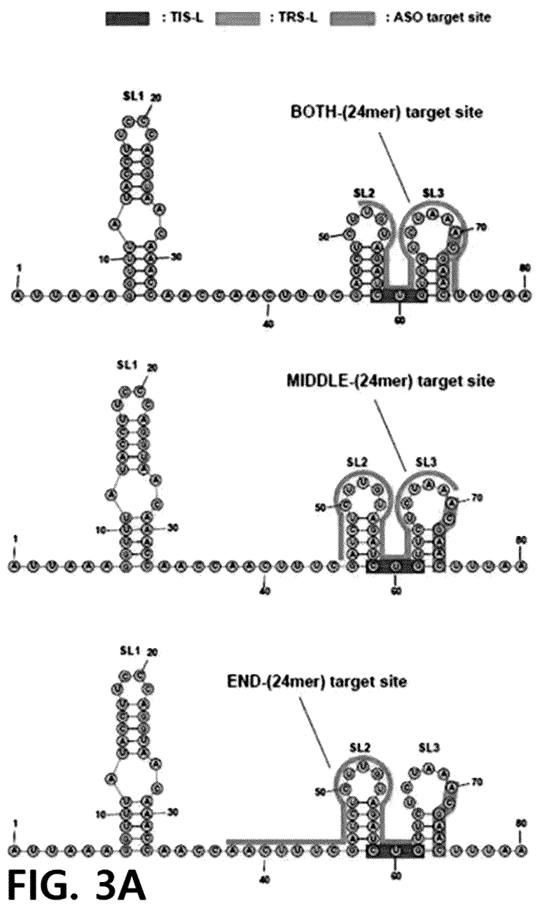

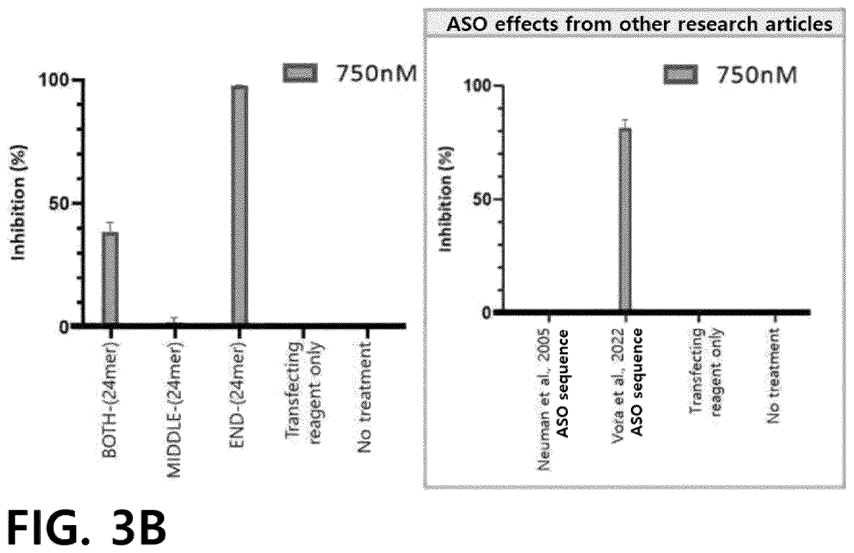

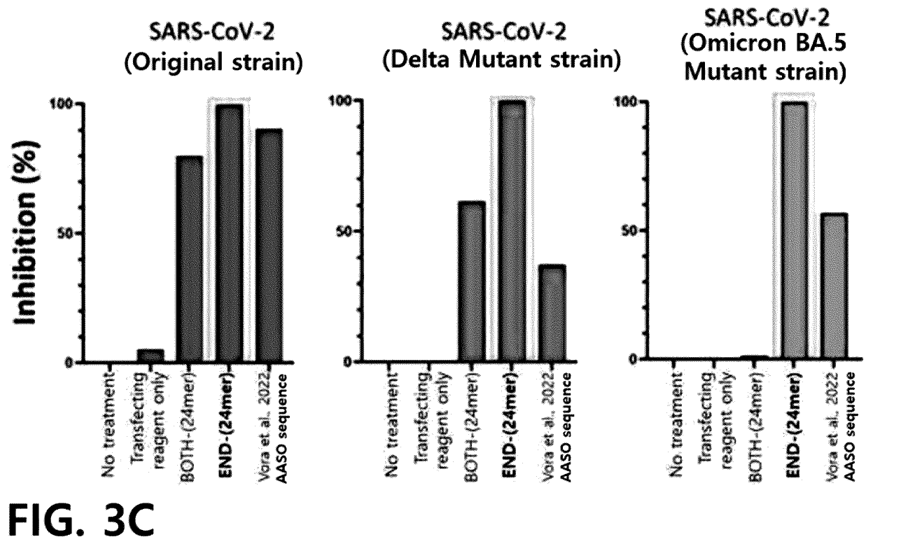

Although it was confirmed that the END-(20 mer) and MIDDLE-(20 mer), which are the typical length of antisense oligonucleotides of 20 mer, did not effectively inhibit the viral replication of SARS-COV-2, the inventors of the present disclosure additionally constructed three 24 mer antisense oligonucleotides targeting TIS-L at different locations by assuming that TIS-L may be a target of antisense oligonucleotides that inhibit the viral replication of SARS-COV-2 (FIG. 3A).
BOTH-(24 mer) Sequence (SEQ ID NO: 5): +G+T+TCGTTTAGAGAACAGATCT+A+C+A [Manufacturer: BIONEER]
MIDDLE-(24 mer) sequence (SEQ ID NO. 6): +T+T+AGAGAACAGATCTACAAGA+G+A+T [Manufacturer: BIONEER]
END-(24 mer) sequence (SEQ ID NO: 7): +C+A+GATCTACAAGAGATCGAAA+G+T+T [Manufacturer: BIONEER]

As a result, as shown in FIG. 3C, the MIDDLE-(24 mer) sequence with a complementary binding site to TIS-L located in the middle did not inhibit the viral replication of SARS-COV-2 at all, whereas it was shown that the BOTH-(24 mer) antisense oligonucleotides targeting both TIS-L and TRS-L and the END-(24 mer) antisense oligonucleotides with a complementary binding site to TIS-L located at the 5′ end inhibited the viral replication of SARS-COV-2. Among these, it was confirmed that in the case of the END-(24 mer) antisense oligonucleotide, the effect of inhibiting the viral replication of SARS-COV-2 was particularly excellent.

Meanwhile, antiviral efficacy against the original strain virus of SARS-COV-2 and mutant strain viruses (Delta mutant, Omicron BA.5 mutant) was additionally evaluated by using the BOTH-(24 mer) antisense oligonucleotide and the END-(24 mer) antisense oligonucleotide. The SARS-COV-2 original strain (NCCP no. 43326), Delta mutant strain (B.1.617.2; NCCP no. 43390) and Omicron mutant strain (BA.5; NCCP no. 43426) viruses used in this experiment were obtained through Korea Disease Control and Prevention Agency's National Culture Collection for Pathogens (NCCP).

As a result, as shown in FIG. 3C, it was confirmed that only the END-(24 mer) antisense oligonucleotide exhibited antiviral efficacy against both of the original strain SARS-COV-2 virus and the mutant strain viruses. In addition, it was confirmed that it exhibited much more superior antiviral efficacy against both of the original strain virus and mutant strain viruses than the antisense oligonucleotide of Vora (Proceedings of the National Academy of Sciences 119.9 (2022): e2117198119), which was previously used as a positive control.

### Example 4. Determination of IC₅₀ of END-(24 Mer)

After treating cells with the END-(24 mer) antisense oligonucleotide from high concentration (1,500 nM) to low concentration (0.98 nM), they were infected with Omicron mutant strains (BA.1, BA.5, XBB.1) and the original strain, and the efficacy of inhibiting the viral replication of viruses was verified through qRT-PCR. The experimental method was the same as described in FIG. 1B. S type or SARS-COV-2 original strain (NCCP no. 43326), Omicron mutant strain (BA.1; NCCP no. 43408), Omicron mutant strain (BA.5; NCCP no. 43426) and XBB.1 mutant strain (XBB.1; NCCP no. 43428) viruses that were used in this experiment were obtained through Korea Disease Control and Prevention Agency's National Culture Collection for Pathogens (NCCP). IC₅₀ values were derived as described in Bae & Lee (Biomolecules & Therapeutics 2021; 29 (3): 268-272).

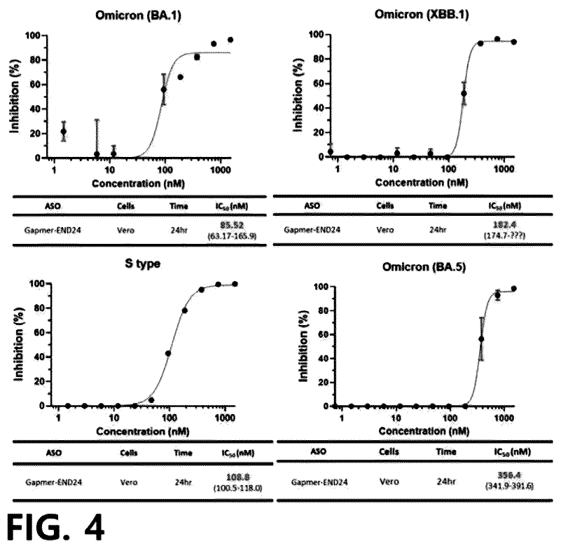

As a result, as shown in FIG. 4, it was confirmed that END-(24 mer) had a very low 50% inhibitory concentration (IC₅₀) in the original strain and all mutant strains (85.52 to 356.4 nM).

### Example 5. Cytotoxicity Test of END-(24 Mer)

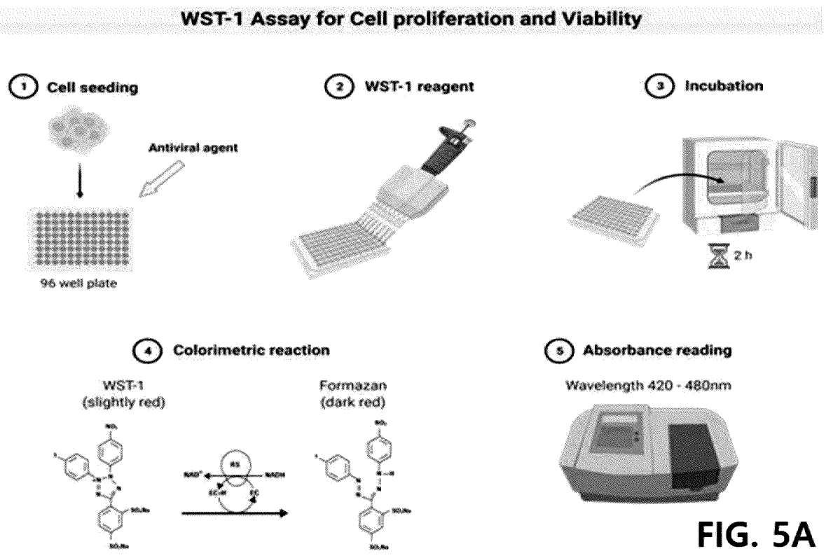

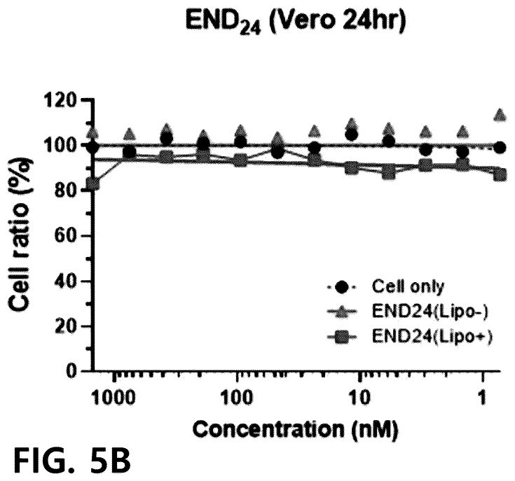

Vero cells were treated with the END-(24 mer) antisense oligonucleotide at different concentrations from high concentration (1,500 nM) to low concentration (0.98 nM), and the survival rate of cells was analyzed by performing WST-1 assay. WST-1 assay was performed by using WST-1 reagent (ROCHE) according to the manufacturer's instructions (FIG. 5A).

As a result, as shown in FIG. 5B, it was confirmed that the END-(24 mer) antisense oligonucleotide was not toxic to Vero cells even at a high concentration of 1,500 nM.

### Example 6. In Vivo Evaluation of Antiviral Therapeutic Effect

### Mouse

BALB/c (7 weeks old, female)
Mock (3), Virus only (24), Virus+Delivery Reagent (24), Virus+Delivery Reagent+ASO (24)

### ASO Sequence

END-(24 mer) sequence (SEQ ID NO: 7): +C+A+GATCTACAAGAGATCGAAA+G+T+T [Manufacturer: IDT]
In the examples, +A, +T, +G, and +C represent LNA, and A, T, C and G represent DNA.

### Virus: SARS-COV-2 Omicron (NCCP43408, BA.1)

ASQ: END-(24 mer)
Delivery Reagent: in vivo-jetPEI® (Polyplus)
DPI: day post infection,
HPI: hours post infection

Using in vivo-jetPEI® (Polyplus) as a RNA/DNA delivery reagent, the in vivo efficacy of END-(24 mer) as a therapeutic agent was evaluated. Following infection of BALB/c mice (KOATECH) with the SARS-COV-2 Omicron (BA.1 variant, input: 1.0×10⁴ pfu/30 ul), a predetermined dose (20 μg) of ASO was administered intravenously using Polyplus's in vivo-jetPEI® transfection reagent once daily from the day of infection (2 hours post-infection) to 3 days post-infection according to the manufacture's instruction. After the infection, the nasal turbinates and lungs were excised and homogenized with 1 mL of Dulbecco's Modified Eagle Medium (DMEM) using TissueLyser (Qiagen) for 1.5 minute. Then, the homogenate was centrifuge for 10 minutes at 13,000 rpm, 4° C., and the supernatant was collected. Viral titers were quantified using a plaque assay.

For plaque assay, after infecting Vero cells that were laid as a monolayer on a 6-well plate with a diluted supernatant, it was covered with DMEM-F12 (Sigma-Aldrich) medium supplemented with 2% agarose, and after 72 hours, it was stained with crystal violet (Georgia Chemicals Inc.). The plaque assay experimental methods were performed as described in Bae & Lee (Biomolecules & Therapeutics 2021; 29 (3): 268-272).

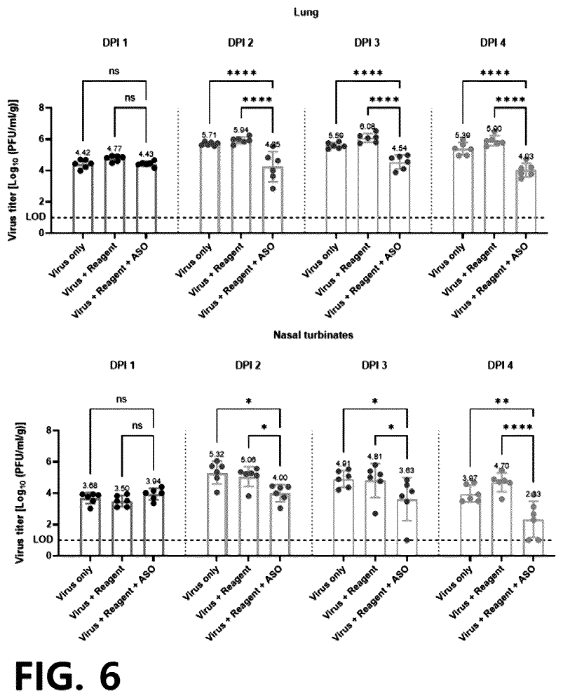

As a result, as shown in FIG. 6, the Virus+Reagent+ASO group exhibited significant inhibition of SARS-COV-2 replication in the lungs and nasal turbinates compared to the Virus+Reagent group. That is, ASO END-(24 mer) demonstrates excellent antiviral efficacy, and these results indicate ASO (END-(24 mer)) as a pharmaceutical composition for the treatment of COVID-19.

As the specific parts of the present disclosure have been described in detail above, it will be clear to those skilled in the art that these specific descriptions are merely preferred exemplary embodiments and do not limit the scope of the present disclosure. Accordingly, the actual scope of the present disclosure will be defined by the appended claims and their equivalents.

What is claimed is:

1. An oligonucleotide represented by the nucleotide sequence of SEQ ID NO: 5 or SEQ ID NO: 7.

2. The oligonucleotide of claim 1, wherein the oligonucleotide is for inhibiting the viral replication of SARS-COV-2.

3. The oligonucleotide of claim 1, wherein the oligonucleotide is an antisense oligonucleotide that targets TIS-L.

4. The oligonucleotide of claim 1, wherein the oligonucleotide represented by the nucleotide sequence of SEQ ID NO: 7 exerts an inhibitory effect on the viral replication of the original strain and mutant strains of SARS-COV-2.

5. The oligonucleotide of claim 1, wherein the oligonucleotide consists of DNA nucleotides, RNA nucleotides, PNA nucleotides, LNA nucleotides, PMO nucleotides, PS nucleotides, 2′-O-Me nucleotides, 2′-O-MOE nucleotides or any combination thereof.

6. The oligonucleotide of claim 5, wherein the oligonucleotide consists of a combination of DNA nucleotides and LNA nucleotides.

7. The oligonucleotide of claim 6, wherein in the oligonucleotide, the 1^{st} to 3^{rd} nucleotides and the 22^{nd} to 24^{th} nucleotides of SEQ ID NO: 5 or SEQ ID NO: 7 are LNA, and the 4^{th} to 21^{st} nucleotides are DNA.

8. A method for preventing or treating COVID-19, comprising administering the oligonucleotide according to claim 1 or a pharmaceutically acceptable salt of the oligonucleotide to a subject in need thereof.

9. The method of claim 8, wherein the oligonucleotide or the pharmaceutically acceptable salt of the oligonucleotide is administered via a carrier.

10. The method of claim 9, wherein the carrier is selected from the group consisting of viral vectors, non-viral vectors, liposomes, cationic polymers, micelles, emulsions, solid lipid nanoparticles and exosomes.
